(19) United States
(12) Patent Application Publication
(10) Pub. No.: US 2023/0014833 A1
(43) Pub. Date: Jan. 19, 2023

(54) COMFORT MANAGEMENT SYSTEM, IN PARTICULAR FOR A MOTOR VEHICLE

(71) Applicant: Valeo Systemes Thermiques, Le Mesnil-Saint-Denis (FR)

(72) Inventors: Alcina Tanghe, Le Mesnil-Saint-Denis (FR); Georges De Pelsemaeker, Le Mesnil-Saint-Denis (FR); Mateo Lopez, Le Mesnil-Saint-Denis (FR); Laurence Cornu, Le Mesnil-Saint-Denis (FR); Olivia Da Silva, Le Mesnil-Saint-Denis (FR)

(73) Assignee: Valeo Systemes Thermiques, Le Mesnil-Saint-Denis (FR)

(21) Appl. No.: 17/785,953

(22) PCT Filed: Dec. 7, 2020
(86) PCT No.: PCT/FR2020/052292

§ 371 Date: Jun. 16, 2022

(30) Foreign Application Priority Data
Dec. 18, 2019 (FR) FR1914737

(51) Int. Cl. B60H 3/00; A61L 9/12

(52) U.S. Cl. CPC B60H 3/0085; B60H 3/0007; A61L 9/12

(57) ABSTRACT

The invention relates to a comfort management system (1), in particular for a motor vehicle, said system being designed to diffuse, into a passenger compartment, a volatile substance designed to neutralize or attenuate an existing olfactory perception that a person could have in this passenger compartment.

The present invention relates to a comfort management system, particularly for a motor vehicle.

Patent application WO2018/133933 describes a predictive perfume control system and associated method which are intended to disperse a perfume into the interior of a motor vehicle on the basis of a feedback system equipped with a decision unit and with a collection of predefined user settings. The dispersion of perfume or scent into the vehicle interior may be based on a predictive user behavioural profile.

In general, with the emergence of new uses of motor vehicles, for example autonomous motor vehicles, car-pooling, car-sharing, it is important that unpleasant odors should not be left in the motor vehicle and passed on from one driver to another. These bad odors are, for example, cigarette smells, animal smells, the smell of vomit, or the like.

Patent application WO2013035070 describes olfactory formulations.

The invention aims to improve this type of situation.

One subject-matter of the invention is thus a comfort management system, particularly for a motor vehicle, this system being designed to diffuse into a vehicle interior a volatile substance that is designed to neutralize or attenuate the olfactory perception that an individual may have when entering this vehicle interior.

Note that the invention differs from a conventional system for automatically improving sensory comfort. In such conventional systems, one or more odor(s) is (are) added to the odor that is to be masked and it may prove to be the case that the mixture of the odors is even more unpleasant than the odors that were to be masked.

The invention notably seeks to add an odor-neutralization step before diffusing the new odor desired or expected by the passenger(s).

According to one of the aspects of the invention, the volatile substance is a blank odor chosen to give the individual in the vehicle interior the sensation of an absence of odor. In particular, this volatile substance contains a plurality of compounds which are selected for their action on the human cells associated with the sense of smell in order to produce this sensation of an absence of odor. In particular, the blank odor mixes with the odor that is to be eliminated/replaced.

The use of a blank odor, particularly in place of masking the unpleasant odors, which is what is being proposed now, with the need to identify the unpleasant odor and allow the counter-odor to diffuse, means that the perception of the odor that is to be masked can be blocked notably by saturating the olfactory receptors. There is thus no need to identify the unpleasant odor in order to correct it. This approach is notably effective for all odors, unpleasant or otherwise.

According to one of the aspects of the invention, the volatile substance is a reset odor perceptible to the individual and triggering in this individual a mental image associated with the odor.

According to one of the aspects of the invention, this reset odor is chosen so that it generates a mental image that is strong enough to attenuate or eliminate the individual's perception of odors existing in the vehicle interior.

According to one of the aspects of the invention, this reset odor is chosen so that it generates a mental image that is strong enough to replace the individual's olfactory perception with another olfactory perception.

According to one of the aspects of the invention, this reset odor is the smell of ground coffee or the smell of ginger. Other odors may be proposed.

In perfumeries, when a great many perfumes are being tested, the vendor gives out a pot of ground coffee which releases a universal odor well known to all. Thus, when ground coffee is smelled, the brain associates a neutral image that in some way eliminates the images held from the previous perfumes.

According to one of the aspects of the invention, the system is designed to diffuse the volatile substance intended to neutralize or attenuate the olfactory perception of the individual in anticipation, before the individual enters the vehicle interior. This individual, on entering the vehicle interior, discovers the olfactory environment and it is important for this individual not to have an unpleasant sensation, which they may have in car-sharing situations.

The invention thus allows the olfactory environment to be prepared before the passenger gets into the vehicle.

The invention also allows the possibility of changing the olfactory ambience during the use of the vehicle on a journey, if the passenger(s) so wish.

According to one of the aspects of the invention, the system is designed so that in a first step it diffuses the volatile substance designed to neutralize or attenuate the olfactory perception of the individual, and in a second step, after the first, it diffuses into the vehicle interior a pleasant volatile substance, notably a fragrance, a perfume, an aroma.

According to one of the aspects of the invention, the system is designed to recognize the individual(s) in the vehicle interior and to diffuse the pleasant volatile substance according to this or these individual(s).

This recognition is notably performed by an exchange of information between the vehicle and the individual. As an alternative, this recognition is obtained using a sensor placed in the vehicle interior and designed to acquire data regarding the individual in the vehicle interior, this sensor notably being a camera.

As an alternative, the system comprises a man-machine interface designed to allow the individual to input a choice of pleasant volatile substance.

According to one of the aspects of the invention, the volatile substance designed to neutralize or attenuate the olfactory perception that an individual may have when entering this vehicle interior is chosen independently of potential undesirable residual odors in the vehicle interior. Thus, the choice of this volatile substance designed to neutralize or attenuate the olfactory perception that an individual may have when entering this vehicle interior need not be made in a complex way, like a choice that is dependent on the undesirable odors.

According to one of the aspects of the invention, the system comprises one or more diffusers for diffusing the volatile substance(s).

According to one of the aspects of the invention, the system comprises one or more sensors designed to be able to recognize passengers installed in the vehicle interior and act according to the data associated with these passengers in order to manage the comfort according to the invention.

According to one of the aspects of the invention, the system comprises a man-machine interface to allow the passenger to choose the olfactory environment in the vehicle interior.

The system comprises a processing unit designed to produce, on the basis of acquired data pertaining to the passenger and using the diffusion of volatile substances, a customized olfactory environment tailored to that passenger.

Another subject-matter of the invention is a comfort management method, particularly for a motor vehicle, this method being designed to diffuse into a vehicle interior a volatile substance that is designed to neutralize or attenuate the olfactory perception that an individual may have when entering this vehicle interior.

According to one of the aspects of the invention, the method involves a first step of diffusing the volatile substance designed to neutralize or attenuate the olfactory perception of the individual, and a second step, after the first, of diffusing into the vehicle interior a pleasant volatile substance, notably a fragrance, a perfume, an aroma

According to one of the aspects of the invention, at least the first step is implemented in the time interval between one passenger leaving the motor vehicle and a new passenger entering this vehicle, notably the first and second steps being implemented in this time interval.

In another exemplary embodiment of the invention, the first and second steps are implemented during a journey in the motor vehicle, notably when the passenger or passengers desire a change in olfactory ambience.

The invention allows the vehicle comfort to be preconditioned from an olfactory standpoint notably by neutralizing the occupant's olfactory perception and by preparing the vehicle interior according to the olfactory demands of the occupant.

According to one aspect of the invention, it is recommended that the vehicle be preconditioned from an olfactory standpoint by using a blank odor to fool the passenger's olfactory perception.

In such a case, for the passenger, the environment is perceived as odorless.

The invention foresees, as a variant, notably preconditioning the vehicle as perfumed by implementing an odor reset step before diffusing a perfume or aroma expected and/or required by the passenger.

For example, the reset odor may be obtained by using ground coffee as universal odor. That induces a cerebral association between a neutral odor and its representative image, and performs the function of cleaning away all the other odors.

This reset may, if desired, be performed as many times as necessary, for example for preconditioning, for example in the case of two different passengers in the vehicle one after the other, between two different journeys, when odors come in from outside.

By virtue of the invention, it is possible to avoid having to recognize an unpleasant odor and having to diffuse a counter-odor for each different spectrum.

Again by virtue of the invention, it is possible to avoid having unpleasant mixed odors because the invention foresees first of all neutralizing the existing odors in one way or another, and only then diffusing the new odor, perfume or fragrance.

In particular, the preconditioning requires a learning phase in order to define, in a personalized manner, what constitutes the right odor.

According to one aspect of the invention, the attenuation or elimination of existing odors in the vehicle interior is performed by said volatile substance for neutralizing or attenuating the existing olfactory perception and not by drawing in or delivering vehicle-interior air, for example using a blower or by opening the windows.

The invention notably allows the olfactory ambience to be influenced without the need to know the odors present in the vehicle.

As a variant, an odor recognition system is provided and allows a neutralization odor or a blank odor to be diffused when certain conditions are met, for example as commanded by the passenger.

If so desired, the volatile substance designed to neutralize or attenuate existing olfactory perception is emitted and then a signature odor is diffused in order to give a predetermined olfactory ambience which can be customised according to the identity of the passenger.

It will be understood that the features and configurations above are in no way limiting. Further features, details and advantages of the invention will become apparent from the following detailed description and from several exemplary embodiments given as non-limiting examples with reference to the attached schematic drawings, in which:

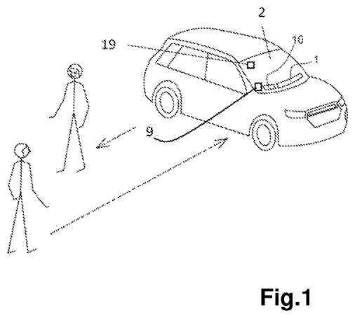

FIG. 1 is a schematic illustration of one exemplary embodiment of a system according to one exemplary embodiment of the invention;

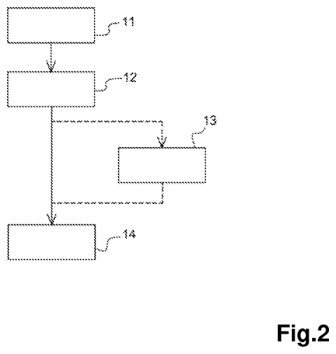

FIG. 2 illustrates various steps of a method according to one example of the invention.

FIG. 1 depicts a comfort management system 1 for a motor vehicle, this system being designed to diffuse into a vehicle interior 2 a volatile substance that is designed to neutralize or attenuate the olfactory perception that an individual may have when entering this vehicle interior.

The system 1 comprises a reservoir 10 of volatile substance.

The volatile substance is a blank odor chosen to give the individual in the vehicle interior the sensation of an absence of odor. In particular, this volatile substance contains a plurality of compounds which are selected for their action on the human cells associated with the sense of smell in order to produce this sensation of an absence of odor. In particular, the blank odor mixes with the odor that is to be eliminated/replaced.

The use of a blank odor, particularly in place of masking the unpleasant odors, which is what is being proposed now, with, in this case, the need to identify the unpleasant odor and allow the counter-odor to diffuse, means that the perception of the unpleasant odor that is to be masked can be blocked notably by saturating the olfactory receptors. There is thus no need to identify the unpleasant odor in order to correct it. This approach is notably effective for all odors, unpleasant or otherwise.

In one variant of the invention, the volatile substance is a reset odor perceptible to the individual and triggering in this individual a mental image associated with the odor. This reset odor is chosen so that it generates a mental image that is strong enough to attenuate or eliminate the individual's perception of odors existing in the vehicle interior. For example, this reset odor is the smell of ground coffee or the smell of ginger. Other odors may be proposed.

The system 1 is designed to diffuse the volatile substance intended to neutralize or attenuate the olfactory perception of the individual in anticipation, before the individual enters the vehicle interior. This individual, on entering the vehicle interior, discovers the olfactory environment and it is important for this individual not to have an unpleasant sensation, which they may have in car-sharing situations.

The system 1 is designed so that, at the moment that a passenger P1 leaves the shared vehicle (step 11 in FIG. 2) there are performed a step 12 of diffusing the volatile substance designed to neutralize or attenuate the olfactory perception of an individual, and a step 13, after the previous step 12, of diffusing into the vehicle interior 2 a pleasant volatile substance, notably a fragrance, a perfume, an aroma, which can be customized to an individual P2 who is to enter the vehicle (step 14).

The individual P2 thus finds themselves in an olfactory environment suited to them, thanks to the olfactory preparation steps 12 and 13.

Thus, steps 12 and 13 are implemented in the time interval between the passenger P1 leaving the motor vehicle and a new passenger P2 arriving in this vehicle.

The olfactory customization step 13 may be omitted, if desired, and then only the odor-neutralizing step 12 is applied.

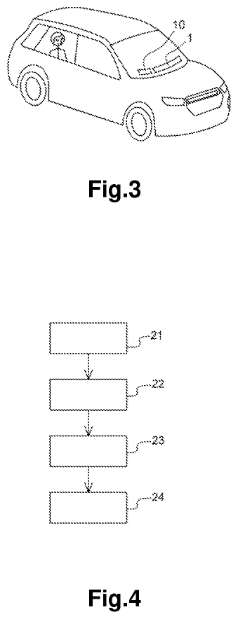

In the example illustrated in FIGS. 3 and 4, the invention also allows the possibility of changing the olfactory ambience during the use of the vehicle on a journey, if the passenger(s) so wish.

In this example, the passenger(s) remains (remain) in the vehicle, for example on a journey. Thus, the system 1 is designed so that, in a step 21, it diffuses a volatile substance, for example a first perfume, and then in a next step 22 of commanding a change in olfactory ambience, then diffuses the volatile substance designed to neutralize or attenuate the olfactory perception of the passenger(s) so as to change the olfactory ambience in the vehicle interior, for example so as to change the ambience following a change in mood of the passenger(s) or by the choice and on the command of the passenger (step 23).

In step 24, a pleasant volatile substance, notably a fragrance, a perfume, an aroma, is diffused into the vehicle interior.

Thus, the passenger(s) can change the olfactory ambience during the course of a journey.

According to one of the aspects of the invention, the system 1 is designed to recognize the individual(s) in the vehicle interior and to diffuse the pleasant volatile substance according to this or these individual(s).

This recognition is notably performed by an exchange of information between the vehicle and the individual. As an alternative, this recognition is obtained using a sensor placed in the vehicle interior and designed to acquire data regarding the individual in the vehicle interior, this sensor notably being a camera.

The system 1 comprises a man-machine interface designed to allow the individual to input a choice of pleasant volatile substance.

The system 1 comprises one or more diffusers 9 for diffusing the volatile substance(s).

The system 1 may comprise one or more sensors 19 designed to be able to recognize passengers installed in the vehicle interior and act according to the data associated with these passengers in order to manage the comfort according to the invention.

The system 1 comprises a processing unit designed to produce, on the basis of acquired data pertaining to the passenger and using the diffusion of volatile substances, a customized olfactory environment tailored to that passenger.

1. A comfort management system for a motor vehicle, the system comprising: a diffuser for diffusing into a vehicle interior a volatile substance that is configured to neutralize or attenuate an existing olfactory perception of an individual inside the vehicle interior.

2. The system as claimed in claim 1, wherein the volatile substance is a blank odor chosen to give the individual in the vehicle interior the sensation of an absence of odor.

3. The system as claimed in claim 1, wherein the volatile substance is a reset odor perceptible to the individual and triggering in the individual a mental image associated with the odor.

4. The system as claimed in claim 3, wherein the reset odor is the smell of ground coffee or the smell of ginger.

5. The system as claimed in claim 1, the diffuser being configured to: diffuse the volatile substance designed to neutralize or attenuate the olfactory perception of the individual, and subsequently diffuse into the vehicle interior a pleasant volatile substance comprising a fragrance.

6. The system as claimed in claim 1, wherein the volatile substance configured to neutralize or attenuate the olfactory perception that the individual has when entering the vehicle interior is chosen independently of potential undesirable residual odors in the vehicle interior.

7. A comfort management method for a motor vehicle, comprising: diffusing into a vehicle interior a volatile substance that is configured to neutralize or attenuate the olfactory perception that an individual has when entering the vehicle interior.

8. The method as claimed in the preceding claim 7, further comprising: subsequent to diffusing the volatile substance configured designed to neutralize or attenuate the olfactory perception of the individual, diffusing into the vehicle interior a pleasant volatile substance comprising a fragrance.

9. The method as claimed in claim 8, wherein at least diffusing of the volatile substance configured to neutralize or attenuate the olfactory perception of the individual is implemented in a time interval between one passenger leaving the motor vehicle and a new passenger entering the motor vehicle.

10. The method as claimed in claim 8, wherein the diffusion of the volatile substance is performed during a journey in the motor vehicle, when the individual inside the vehicle desire a change in olfactory ambience.

11. The method as claimed in claim 8, further comprising:
recognizing the individual in the vehicle interior using a camera; and
diffusing the pleasant volatile substance according to the individual.

12. The system of claim 5, further comprising: a camera configured to recognize the individual and diffuse the pleasant volatile substance according to the individual.

13. The system of claim 5, further comprising: a man-machine interface configured to receive input from the individual regarding a choice of pleasant volatile substance.

14. The system of claim 1, further comprising: a reservoir of the volatile substance operatively connected to the diffuser.
